(19) United States
(12) Patent Application Publication
(10) Pub. No.: US 2008/0219969 A1
(43) Pub. Date: Sep. 11, 2008

(54) Concentrate of Chikungunya-Specific Immunoglobulins as a Medicinal Product

(76) Inventor: Roland Schmitthaeusler, Montigny le Bretonneux (FR)

Correspondence Address: CERMAK KENEALY & VAIDYA LLP, 515 E. BRADDOCK RD, SUITE B, ALEXANDRIA, VA, 22314, US

(21) Appl. No.: 11/912,439

(22) PCT Filed: Apr. 2, 2007
(86) PCT No.: PCT/FR2007/000561

§ 371 Date: May 14, 2008

(30) Foreign Application Priority Data
Mar. 31, 2006 (FR) 0602802

(51) Int. Cl. A61K 39/395; A61P 31/00

(52) U.S. Cl. 424/130.1

(57) ABSTRACT

The invention concerns a new medicinal product for the treatment of chikungunya, i.e a concentrate of chikungunya-specific immunoglobulins, as well as its process of preparation.

The invention concerns a new medicinal product for the treatment of chikungunya, i.e a concentrate of chikungunya-specific immunoglobulins, as well as its process of preparation.

### INTRODUCTION

Chikungunya (abbreviated CHIK), is an infectious tropical disease caused by an arbovirus (an alphavirus of the Togaviridae family), transmitted by mosquitoes of the genus Aedes. The name comes from the Bantu language, and means: he who bends, he who curls up, or bent man's disease because it causes very severe joint pain combined with stiffness, which gives infected patients a very characteristic bent appearance.

Viruses that employ arthropod vectors in their cycle are grouped under the general term arbovirus. Arboviruses are defined by the WHO as viruses that subsist in nature essentially or mostly through biological transmission between susceptible vertebrate hosts by hematophagous arthropods; they multiply and provoke viremia in the vertebrate, proliferate in the tissues of the arthropod and are transmitted to another vertebrate by the biting insect after an extrinsic incubation period.

Transmission of the virus from a viremic host to an adult female mosquito takes place via the blood that is sucked out when the bite occurs. The virus multiplies inside the mosquito, crosses the animal's stomach barrier and is found in the salivary glands. Contamination of a healthy human is achieved by the anticoagulant saliva of the mosquito, released just before the bite into a blood vessel. The window during which a person is a viremic host before falling ill is only a few days.

Out of over 950 species of mosquitoes, several of them are able to transmit chikungunya, but only Aedes aegypti and Aedes albopictus have been identified to date as epidemic vectors, due to their adaptation to areas of human habitation. These same species are also involved in the transmission of other arboviruses: dengue fever, hemorrhaging dengue fever (HDF), yellow fever, etc.

The clinical profile is dominated by a high fever similar to that of dengue (dengue is often mistaken for chikungunya and vice-versa), combined with incapacitating joint pain and sometimes skin rash. However, there are severe forms that have been ignored up to now: fulminating hepatitis, heart attacks, meningoencephalitis, etc. Several other arboviruses of the alphavirus genus (approximately 30-kD capsid and polyadenylated RNA at 3′) such as Ross River, O'nyong-nyong and Mayaro have been associated with similar symptoms.

Incubation of the disease lasts from four to seven days on average. Viremia, the presence of the virus in the blood and therefore of possible transmission, extends over approximately five days. Antibodies then develop. They remain in the blood. Immunity is therefore usually acquired for life, or at least for a year (cf. phase II trial below).

### PRIOR ART

Currently, there exists no virucidal treatment and no vaccine that has received marketing authorization.

Treatment is purely symptomatic, to lower the fever and reduce pain.

A phase I trial and a phase II trial have been conducted in the United States for a chikungunya vaccine by the United States Army Medical Research Institute of Infectious Diseases.

The phase II (Edelman R et al. “Phase II safety and immunogenicity study of live chikungunya virus vaccine” TSI-GSD-218. June 2000; Am J Trop Med Hyg, 62:681-5) randomized, double blind, placebo controlled research consisted of a study of the safety and immunogenicity of a live purified chikungunya (CHIK) vaccine on plate in 73 adult volunteers who were in good health. 59 volunteers were immunized once subcutaneously with the CHIK vaccine and 14 were injected with the placebo. 57 (98%) of the 58 who received the vaccine developed anti-CHIK neutralizing antibodies on day 28, and 85% of vaccinated subjects were still seropositive one year afterward.

The combination of two antiviral compounds, ribavirin and interferon-alpha, has also been tested on chikungunya (Briolant S et al., “In vitro inhibition of Chikungunya and Semliki Forest viruses replication by antiviral compounds: synergistic effect of interferon-alpha and ribavirin combination”, Antiviral Res., February; 2004 61(2):111-7. This combination of IFN-alpha2b and ribavirin presents a synergistic antiviral effect on chikungunya, which is sufficiently promising to consider its use in therapy.

However, such treatment would be extremely expensive and repetitive and would involve the many known side effects of interferon.

### SUMMARY OF THE INVENTION

Faced with this absence of established treatment, a vaccine that will not be ready soon and burdensome antiviral treatments, the Applicant has sought to offer a new treatment against chikungunya.

The Applicant has demonstrated in a surprising manner that administration of a concentrate of chikungunya-specific immunoglobulins can be used to resolve this technical problem.

### DEFINITIONS

The term “concentrate” refers to a product obtained by elimination of certain components. A concentrate of immunoglobulins is obtained by elimination of certain components of the plasma to achieve an immunoglobulin-enriched plasma fraction.

The term “immunoglobulin” (Ig) refers to a natural globulin, present mainly in the plasma, with antibody functions, which can be used in curative or preventive therapy.

Immunoglobulins are heterodimers composed of 2 heavy chains and 2 light chains, linked by disulfide bridges. Each chain is constituted, in N-terminal position, of a variable domain or region (coded for by rearranged V-J genes for the light chain and V-D-J for the heavy chain) that is specific to the antigen against which the antibody is directed, and in C-terminal position, of a constant region, composed of a single CL domain for the light chain or 3 domains (CH1, CH2 and CH3) for the heavy chain. The combination of variable domains and CH₁ and CL domains of the heavy and light chains forms the Fab parts, which are connected to the Fc region by a very flexible hinge region that allows each Fab to bind to the antigen target while the Fc region, which mediates the effector properties of the antibody, remains accessible to effector molecules such as FcγR receptors and C1q.

IgG are the most abundant immunoglobulins (75 to 80% of circulating antibodies). They protect the body against bacteria, viruses and toxins that circulate in the blood and the lymph. In addition, they quickly bind to the complement (one of the components of the immune system). They also participate in memory response, which is the basis of immunity upon which the mechanism of vaccination is founded. Lastly, immunoglobulins G cross the placental barrier and thus produce passive immunity in the fetus.

IgA are mainly found in secretions such as saliva, intestinal juices, sweat and breast milk. The main role of immunoglobulins A is to prevent pathogenic agents from binding to cells, particularly to the protective cells that make up the mucous membranes and epidermis.

IgM are immunoglobulins secreted upon the body's first contact with an antigen. They are the first type of immunoglobulins released by plasmocytes. The presence of IgM in the blood indicates a current infection.

Enzymatic proteolysis of immunoglobulins by papain generates 2 identical fragments, which are known as Fab (Fragment Antigen Binding), and one Fc (crystallizable fraction) fragment. The Fc fragment supports the effector functions of immunoglobulins.

By pepsin proteolysis, an F(ab′)2 fragment is generated, in which the two Fab fragments remain bound by two disulfide bridges, and the Fc fragment is cleaved into several peptides. The F(ab′)2 fragment is formed from two Fab′ fragments (one Fab′ fragment consisting of one Fab and a hinge region), linked by intercatenary disulfide bridges to form an F(ab′)2.

The term “chromatography” refers to a method of separation of the components of a mixture based on their selective adsorption by a suitable medium.

### DETAILED DESCRIPTION OF THE INVENTION

First of all, the invention relates to a concentrate of chikungunya virus-specific immunoglobulins as a medicinal product.

The use of immunoglobulin-enriched human plasma fractions for the treatment of various infections and congenital deficiencies has been known since the development of the ethanol precipitation process by Cohn (Cohn et al. 1946, J. Am. Chem. Soc. 68, 459; Oncley et al. 1949, J. Am. Chem. Soc. 71, 541).

In particular, the concentrate according to the invention is composed of a concentrate of immunoglobulins A, G and M, or a concentrate of immunoglobulins G exclusively, or a concentrate of immunoglobulins M exclusively, specific to the chikungunya virus as a medicinal product.

Particularly preferably, the concentrate according to the invention includes from 90 to 98% of immunoglobulins.

The concentrate according to the invention can contain, in addition to complete chikungunya virus-specific immunoglobulins, chikungunya virus-specific F(ab)′2 and/or Fab fragments, in particular from 5 to 50% F(ab)′2 and/or Fab, in particular at least 50 to 60 g/L of Ig and fragments for a pharmaceutical preparation.

Such F(ab)′2 or Fab fragments, which contain the antibody's binding site, may have lost a certain number of the properties of the whole antibody from which they were derived, such as the ability to bind Fcgamma receptors.

The concentrate according to the invention can contain, in addition to complete chikungunya virus-specific immunoglobulins, chikungunya virus-specific F(ab)′2 or Fab fragments that come exclusively from IgG and IgM.

According to the invention, from 1 to 10 mmol of magnesium and/or zinc could be added to the concentrate.

Another subject of the invention is the use of a concentrate according to the invention for the manufacturing of a medicinal product for the treatment of chikungunya.

This treatment is prophylactic or curative. It is used either to confer passive immunity to persons not yet infected in a region of epidemic, or to care for patients already infected with the virus.

The medicinal product in question is administered by topical, subcutaneous, oral, intramuscular or intravenous route.

It is effective for several weeks, approximately 21 days, and, beyond, this administration must be repeated if the epidemic or symptoms persist.

The invention also concerns a process for preparing a concentrate according to the invention.

The 1^{st} step of this process is the creation of a pool of at least 1000 plasma donations, each donation containing a sufficient titer of anti-chikungunya Ig. These donations come from people who have been in contact with the disease, or patients who have developed the disease.

Titration can be performed according to C. van de Water et al., Journal of Immunological Methods, 166(1993), 157-164.

In order to enrich this plasma pool in immunoglobulins, the other components of the plasma, known as “lipid and protein contaminants” are precipitated in a single step. This purification by precipitation in a single step may take place by diluting the plasma in precipitation conditions according to Steinbuch (Steinbuch M., Archiv. Biochem. Biophys., 134, 279-284) with caprylic acid and by adding caprylic acid. It can also take place through precipitation agents such as Rivanol, aluminum chloride, cetylpyridinium chloride, octanoic acid, polyphosphates and in presence of adsorption agents with tricalcium phosphate and bentonite.

The supernatant can constitute the concentrate of immunoglobulins according to the invention. It therefore contains a mixture of IgG, A and M. This is recovered, for example by centrifugation or filtration, optionally by adding at least one filtration additive.

The supernatant can undergo conventional viral deactivation processing with a solvent/detergent (Triton X100).

If the precipitation was a caprylic precipitation, the residues of caprylic acid in the supernatant are eliminated by PO4 calcium.

In order to obtain a concentrate of IgG, IgA or IgM, the method described in the application EP02727688.0 can be applied. The supernatant then undergoes an additional step of purification by chromatography on an anion exchanger performed in alkaline pH. In particular, the pH of the supernatant is adjusted beforehand to a pH between 8.9 and 9.1, and the column is loaded with a buffer at a pH 8.9 to 9.1. The chromatography step allows the adsorption of immunoglobulins and passage of non-retained proteins into the effluent. Chromatography can be performed on a reticulated polysaccharide or vinyl polymer gel, grafted with DEAE, TMAE or QAE groups.

After washing the column with the same buffer as the loading buffer to eliminate non-retained proteins, immunoglobulins G are eluted with a phosphate buffer, the pH of which is between 4 and 7, preferably at pH 6.2.

An optional subsequent elution with the same phosphate buffer supplemented with 100 to 175 mM NaCl, preferably 150 mM, at a pH of 6 to 6.3, can be used to collect IgA.

An optional subsequent elution with the same buffer adjusted to a pH of 6 to 7 and supplemented with 250 to 350 mM NaCl, preferably 300 mM, can be used to collect IgM.

Any type of mixture between IgA, IgG and IgM can be considered by mixing the concentrates as they are described above.

Immunoglobulins thus eluted and collected can be concentrated by ultrafiltration and put through conventional sterilizing filtration then filtration through nanometric filters with porosity decreasing from 100 to 15 nanometers.

To the solution of concentrated and filtered immunoglobulins is added a pharmaceutically acceptable stabilizer, then it is packaged as a sterile solution and optionally frozen and lyophilized.

Application of nanofiltration makes it possible to eliminate viruses that are resistant to solvent/detergent treatment.

In order to prepare a concentrate of chikungunya virus-specific Ig and F(ab)′2 or Fab fragments, a concentrate of immunoglobulins (1) was prepared: a mixture of A, G and M or a mixture of G and M, or of G only, or of M only, as described above, then, in a second step, a part of the obtained Ig concentrate is digested to obtain F ab)′2 or Fab fragments (2), and, in a third step, concentrates (1) and (2) are mixed.

In order to obtain F(ab)′2 fragments, proteolysis takes place in pepsin 1% by weight of proteins at pH 4 and 35° C., (the IGLOO protocol)

To obtain Fab fragments, proteolysis takes place in papain.

1. A medicinal product comprising a concentrate of chikungunya virus-specific immunoglobulins as a medicinal product.

2. The medicinal product according to claim 1, wherein said immunoglobulins comprise immunoglobins A, G, and M.

3. The medicinal product according to claim 1, wherein said concentrate comprises immunoglobulins G.

4. The medicinal product according to claim 1, wherein said concentrate comprises immunoglobulins M.

5. The medicinal product according to claim 1, comprising from 90 to 98% of immunoglobulins.

6. The medicinal product according to claim 1, further comprising chikungunya virus-specific F(ab)′2 fragments.

7. The medicinal product according to claim 1, further comprising chikungunya virus-specific Fab fragments.

8. The medicinal product according to claim 7, further comprising from 5 to 50% F(ab)′2 and/or Fab.

9. The medicinal product according to claim 6, wherein the F(ab)′2 or Fab fragments are F(ab)′2 or Fab fragments of IgG and IgM.

10. The medicinal product according to claim 1, further comprising from 1 to 10 mmol of magnesium is added thereto.

11. The medicinal product according to claim 1, further comprising from 1 to 10 mmol of zinc is added thereto.

12. A method of making a medicinal product comprising forming a concentrate of chikungunya virus-specific immunoglobulins.

13. The method according to claim 12 wherein said medicinal product is in a form to be administered by a route selected from the group consisting of topical, subcutaneous, oral, intramuscular, and intravenous.

14. A process for preparing a concentrate according to claim 1, wherein said process comprises:
creating a pool of at least 1000 plasma donations, each donation comprising a sufficient titer of anti-chikungunya Ig,
precipitating lipid and protein contaminants in a single step,
recovering an Ig concentrate in the supernatant.

15. A process for preparing a concentrate according to claim 1, wherein said process comprises:
creating a pool of at least 1000 plasma donations, each donation comprising a sufficient titer of anti-chikungunya Ig,
precipitating lipid and protein contaminants in a single step,
chromatographing the supernatant on an anion exchanger in alkaline pH,
eluting IgG with a phosphate buffer at a pH comprised between 4 and 7, preferably at pH 6.2,
optionally, subsequently eluting IgA with the same phosphate buffer further comprising 100 to 175 mM NaCl, preferably 150 mM, at a pH of 6 to 6.3,
optionally, subsequently eluting IgM with the same phosphate buffer further comprising 250 to 350 mM NaCl at a pH between 6 and 7,
optionally, mixing IgG, IgA, and IgM concentrates.

16. The process according to claim 15, wherein the pH of the supernatant is adjusted to between 8.9 and 9.1 and the chromatography column is loaded with a buffer at a pH 8.9 to 9.1 before chromatography.

17. A process for preparing a concentrate of chikungunya virus-specific F(ab)′2 fragments, comprising
(1) preparing a concentrate of Ig, IgG, or IgM according to claim 15,
(2) subjecting a part of the earlier concentrate to proteolysis to obtain F(ab)′2 or Fab fragments,
(3) mixing fractions (1) and (2).

18. A Process for preparing a concentrate of chikungunya virus-specific F(ab)′2 fragments of IgG and IgM, comprising:
(1) preparing a concentrate of IgG according to claim,
(2) preparing a concentrate of IgM according to claim 15,
(3) mixing fractions (1) and (2),
(4) subjecting a part of the earlier mixture to proteolysis to obtain F(ab)′2 or Fab fragments of IgG and IgM,
(5) mixing (3) and (4).

19. The process according to claim 17, wherein the proteolysis takes place in pepsin 1% by weight of proteins at pH 4 and 35° C. to obtain F(ab)′2 fragments.

20. The process according to claim 17, wherein the proteolysis to obtain Fab fragments takes place in papain.

21. The process according to claim 14, wherein the precipitation is a caprylic precipitation, and wherein in residues of caprylic acid in the supernatant are eliminated by PO4 calcium.

22. The process according to claim 14, wherein the precipitate is separated by filtration after addition of at least one filtration additive.

23. The process according to claim 14, wherein the supernatant is treated with a solvent/detergent.

24. The process according to claim 15, wherein the eluted immunoglobulins are concentrated by ultrafiltration and put through conventional sterilizing filtration then filtration through nanometric filters with porosity decreasing from 100 to 15 nanometers.

25. The process according to claim 15, wherein the solution of concentrated and filtered immunoglobulins has a pharmaceutically acceptable stabilizer added to it, then it is packaged as a sterile solution and optionally frozen and lyophilized.
